(19) United States
(12) Patent Application Publication
(10) Pub. No.: US 2014/0179786 A1
(43) Pub. Date: Jun. 26, 2014

(54) Treatment of Alzheimer's Disease Using Tolfenamic Acid

(71) Applicant: Board of Governors for Higher Education, State of Rhode Island and Providence Plantations, Providence, RI (US)

(72) Inventor: Nasser Zawia, Warwick, RI (US)

(73) Assignee: Board of Governors for Higher Education, State of Rhode Island and Providence Plantations, Providence, RI (US)

(21) Appl. No.: 14/136,208

(22) Filed: Dec. 20, 2013

Related U.S. Application Data
(60) Provisional application No. 61/739,930, filed on Dec. 20, 2012.

(51) Int. Cl. A61K 31/196

(52) U.S. Cl. CPC A61K 31/196
USPC 514/567

(57) ABSTRACT

A method is disclosed of reducing the level of tau protein in a subject's brain for the treatment of tau tangles. The method includes administrating an effective dose of drug that reduced Sp1 levels to the subject to lower tau mRNA and protein levels, CDK5 mRNA and protein levels, as well phosphorylated tau in the brain.

### PRIORITY

The present application claims priority to U.S. Provisional Patent Application Ser. No. 61/739,930 filed Dec. 20, 2012, which is hereby incorporated by reference in its entirety.

### BACKGROUND

The number of people suffering from Alzheimer's disease (AD) is expected to increase exponentially in the coming decades. AD is the third most expensive disease in the US estimated to cost the economy about $100 billion annually. In addition to AD many other neurodegenerative diseases such as Parkinson's disease and taupathies impose a great burden on our healthcare system. Standard therapeutic approaches have failed and there are no disease modifying drugs available in the market.

Alzheimer's Disease is the most prevalent form of senile dementia. One hundred years after the discovery of AD, the etiology of this disease still remains unknown and although a few drugs have been approved, their benefits have been extremely modest. Advances in basic research have identified many pathways that lead to the formation of key pathological features of AD, Amyloid plaques (amyloidogenesis), and abnormal twisted forms of the protein tau (tangles). The available limited treatment strategies have mostly focused on targets associated with the production of the amyloid (Aβ) peptides which aggregate into various assemblies and their deposits from the core of the plaque.

AD is characterized by chronic and progressive loss of neurons in discrete areas of the brain, causing debilitating symptoms such as dementia, loss of memory, and eventually, premature death. Currently five drugs are approved for AD in the US. With exception of Memantine, all are cholinesterase inhibitors designed to build up the levels of the neurotransmitter acetylcholine, which are low due to the early loss of neurons that contain this neurotransmitter. On the other hand, Memantine prevents over-stimulation of the NMDA type of glutamate receptors which contribute to the pathogenesis of several neurodegenerative diseases. All of the approved drugs are not disease modifying, but have shown very modest improvements in behavioral and functional measures in a subset of patients.

The general emphasis of currently available drugs has been to ameliorate the pathogenicity of Aβ peptides by reducing Aβ production through inhibiting the secretases that process Aβ, enhancing Aβ clearance by immune mechanisms, and disrupting Aβ aggregation using small molecules. Other investigative drugs target the overall process of neurodegeneration by combating oxidants or preventing cell loss. Furthermore several off-the-shelf drugs such as the non-steroidal anti-inflammatory drugs (NSAIDS) and cholesterol lowering drugs are being tested for their ability to decrease the risk associated with AD through their anti-inflammatory properties.

There remains a need therefore, for disease modifying drugs for treatment of neurodegenerative diseases and for effectively treating these neuridegenerative diseases such as Alzheimer's disease and Parkinson's disease.

### SUMMARY

In accordance with an embodiment, the invention provides a method of reducing the level of tau protein in a subject's brain for the treatment of tau tangles. The method includes administrating an effective dose of drug that reduced Sp1 levels to the subject to lower tau mRNA and protein levels, CDK5 mRNA and protein levels, as well phosphorylated tau in the brain.

### BRIEF DESCRIPTION OF THE DRAWINGS

The following description may be further understood with reference to the accompanying drawings in which:

FIG. 1 shows a proposed mechanism for tolfenamic acid-induced down-regulation of the tau gene in accordance with an embodiment of the present invention;

FIGS. 2A and 2B: show that tolfenamic acid treatment has been found to reduce total cortical tau gene and protein expression in mice;

FIGS. 3A and 3B show CDK5 protein and gene expression after tolfenamic acid treatment;

FIGS. 4A and 4B show levels of tau phosphorylated at Ser 235 and Thr 181 after tolfenamic acid treatment.

FIGS. 5A and 5B shows memory improvement in homozygous APP YAC transgenic mice; and

FIG. 6 shows working memory improvement in homozygous APP YAC transgenic mice.

### DETAILED DESCRIPTION

It has been discovered that tolfenamic acid lowers the levels of proteins associated with AD pathogenesis and taupathies. This is a mechanism-based drug that is disease modifying. The characteristic neuropathological deposits identified in Alzheimer's disease (AD) are the extracellular amyloid beta (Aβ) plaques and intracellular tau tangles. Neurofibrillary degeneration in the absence of β-amyloid, is also seen in several tauopathies such as Guam parkinsonism dementia complex, dementia pugilistica, corticobasal degeneration, Pick's disease, frontotemporal dementia and parkinsonism linked to chromosome 17 (FTDP-17), and progressive supranuclear palsy (PSP). It is well established that hyperphosphorylation of tau is responsible for the neurofibrillary lesions found in these conditions, while Aβ, a cleavage product of the amyloid precursor protein (APP), is a main constituent of the plaques. Studies have also shown that reduction in wild-type tau prevents Aβ-dependent behavioral and cognitive deficits (Roberson et al., 2007), suggesting that therapeutic interventions that alter the levels of tau may be beneficial.

The transcription factor specificity protein 1 (Sp1) is essential for the regulation of the tau gene and studies from our lab have provided convincing evidence that either silencing of the Sp1 gene or treatment of animals with tolfenamic acid lowers the expression of AD-related Sp1 target genes. Data from our lab has demonstrated that tolfenamic acid can reduce tau protein and mRNA levels in animal models. It also downregulated the kinase involved in tau phosphorylation, CDK5, and decreased the levels of two phosphorylated forms of tau at serine 235 and threonine 181. Furthermore, it was found that such treatment improves the cognitive performance of animal models of neurodegenerative diseases. Thus it is believed that tolfenamic acid and derivatives thereof may be used to treat patients that suffer from these neurodegenerative diseases.

During the past 20 years, drug discovery has focused on targeting intermediates mentioned in the amyloid hypothesis of AD, and so far no successful disease-modifying candidate has been found. Much less attention was paid to tau which is abnormally hyperphosphorylated and forms aggregates. Tolfenamic acid, a drug already available in the European market for the management of migraine headaches, represents a novel class of drugs that could be used due to its unique ability to promote the degradation of specificity protein 1 (Sp1), a transcription factor involved in AD pathology and whose mRNA and protein levels are elevated within the frontal cortex of AD patients as well as in animal models with AD-like pathology.

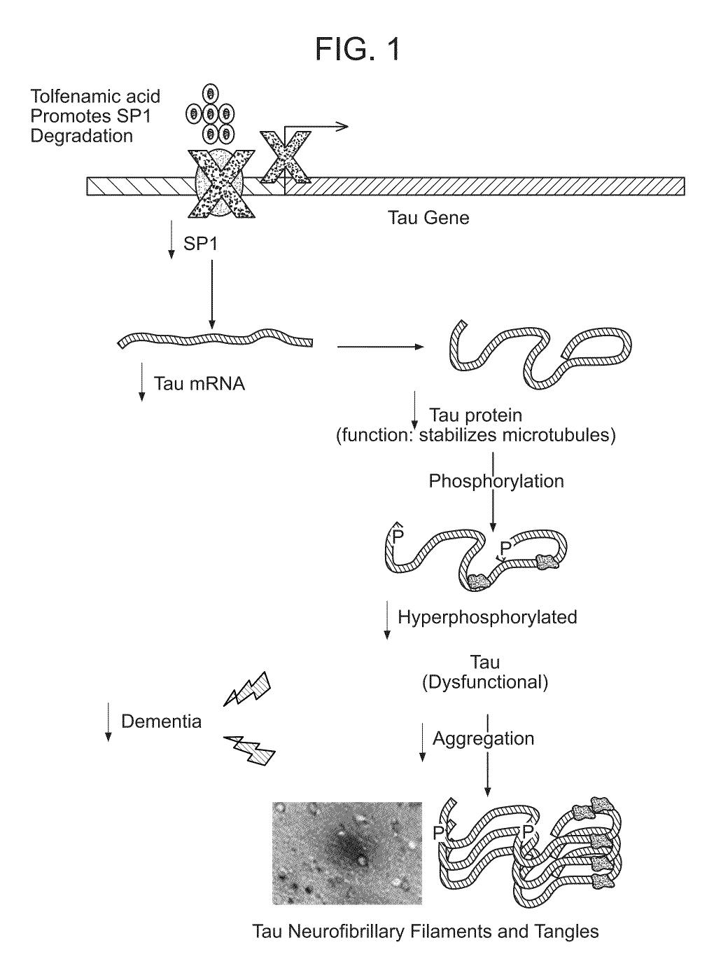

With reference to FIG. 1, it has been discovered that silencing of the Sp1 gene dramatically reduces the APP promoter activity. In addition, Sp1 regulates the expression of tau and any mutations on the Sp1 binding regions on the tau promoter decrease tau expression. Furthermore, Sp1 levels were found to be elevated in the frontal cortex of AD patients as well as in transgenic animals that develop AD-like pathology. Therefore, targeting Sp1 is an ideal approach to lower total tau levels, and such reduction is likely to impact any post-translational modifications of tau and thus provide an alternative therapeutic approach to be used in AD and other tauopathies.

In accordance with the present invention and with reference to FIG. 1, it has been discovered that tolfenamic acid promotes the degradation of the transcription factor Sp1 that significantly decreased tau transcription/translation, downregulated the kinase involved in tau phosphorylation, CDK5, and also significantly lowered levels of tau and phospho-tau (P-tau). These changes are expected to ultimately reduce the tangle burden and pathology in AD. For the first time and quite surprisingly, administered tolfenamic acid was demonstrated not only to significantly improve learning and memory, but more importantly it impacted working memory function. It is expected that tolfenamic acid analogs and anthranylic acid derivatives or other drugs reducing Sp1 levels may have a similar effect in reducing symptoms of AD and taupathies.

### EXAMPLE 1

### Tolfenamic Acid Towers Tau in an APP Knock-Out Mouse Model

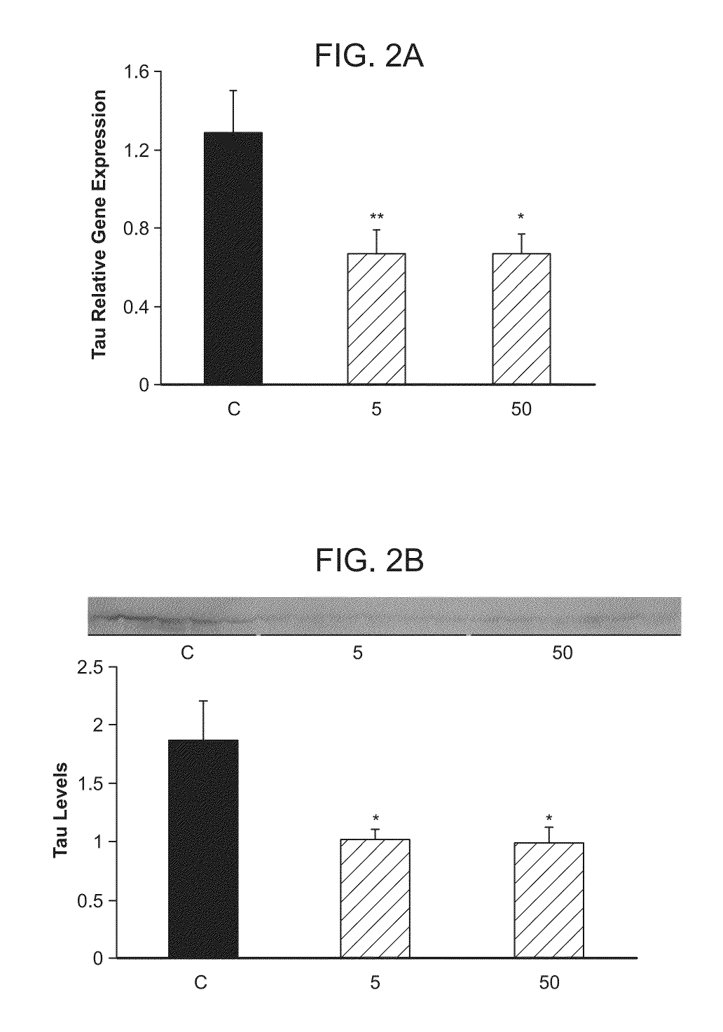

It has been discovered that tolfenamic acid lowers the levels of the transcription factor Sp1, and consequently reduce the levels of an Sp1 target gene, APP, along with its pathogenic cleavage product Aβ. It was expected that since Sp1 regulates tau, tolfenamic acid could significantly lower tau levels. Therefore, in the study design 0, 5 and 50 mg/kg/day tolfenamic acid was administered for 34 days. Surprising, it was demonstrated that tau mRNA expression was significantly lowered by 48%. To assess whether lowered mRNA expression, total tau protein levels were measured and were also found to be significantly reduced by 46%, p<0.05, as shown, for example in FIGS. 2A and 2B.

FIG. 2A shows the relative tau mRNA expression and FIG. 2B shows the total tau protein levels in control (C) (n=5) and tolfenamic acid treated animals (5 or 50 mg/kg/day) (n=6 in each group). Total mRNA levels were analyzed by real time PCR with β-actin as endogenous control. Protein expression was measured by Western blot analysis and quantified as a ratio to the house keeping protein β-actin (antibodies: T9450, Sigma-Aldrich, MO; 4970, Cell Signaling, MA). The insert in FIG. 2B shows control and treatment of tau Western blot bands. Values shown are the mean ±SEM. The symbol “*” indicates that values are significantly different from control as determined by a Student's t-test (p<0.05).

### EXAMPLE 2

### Tolfenamic Acid Reduces CDK5 Protein and Gene Expression Levels

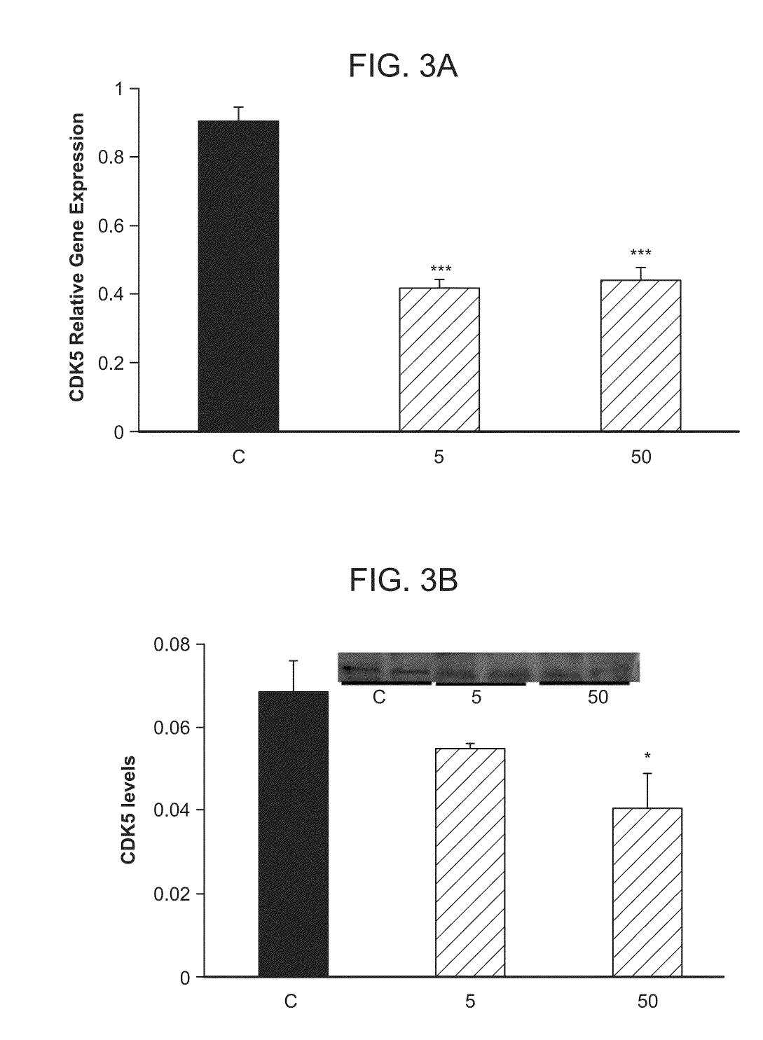

Since Sp1 also regulates CDK5 activators, the effects of tolfenamic acid on CDK5 were also tested. It was found that daily administration of tolfenamic acid to mice for a month lowered the gene expression of CDK5 in the cerebral cortex by about 50% as shown, for example, in FIG00. 3A (using one-way ANOVA F(2,13)=59.647, p=2.8×10⁻⁷ Tukey-Kramer post-test p<0.05 for the C vs the 5 mg/kg group and for the C vs the 50 mg/kg group). There was a lowering trend in CDK5 levels (shown in FIG. 3B) that was not significant when analyzed with one-way ANOVA (F(2,8)=4.086, p=0.059). When comparing the 50 mg/kg dose group to the control however, by Tukey-Kramer test, the 40% lowering in CDK5 from control was statistically significant (p<0.05).

### EXAMPLE 3

### Tolfenamic Acid Reduces the Expression of Phosphorylated Tau

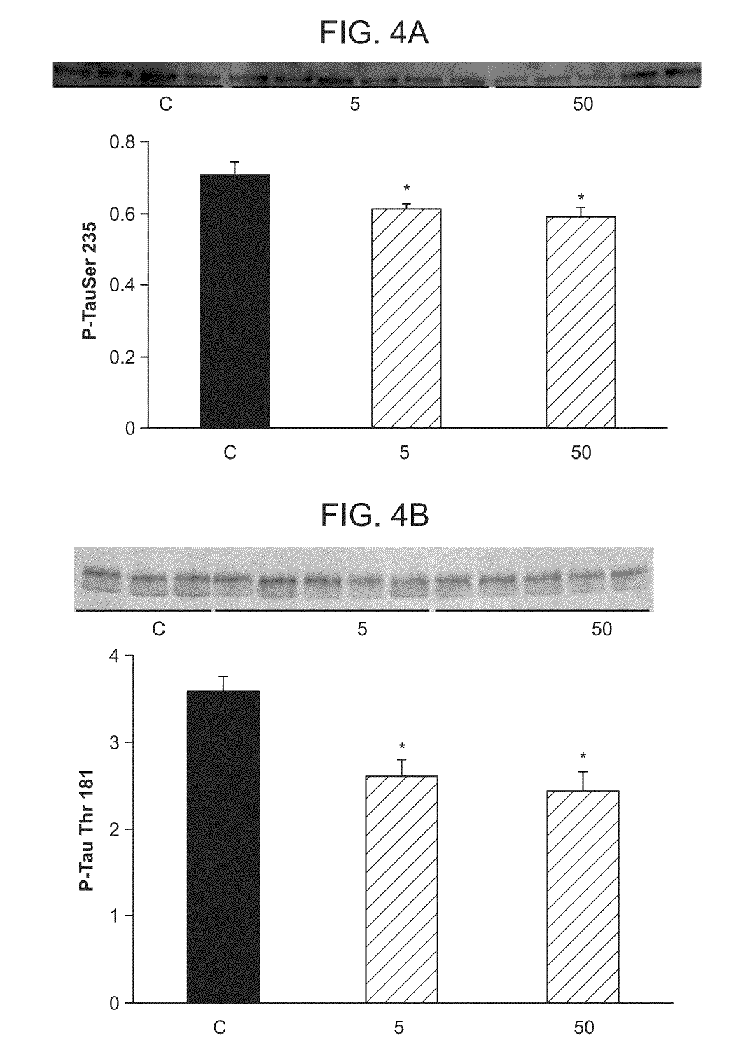

As phosphorylation of tau affects its function and its ability to bind to microtubules, it was important to test how phosphorylated tau is affected by the treatment. P-tau levels were analyzed by Western blotting using specific antibodies. P-tau at Ser 235 (as shown in FIG. 4A) and P-tau at Thr 181 (as shown in FIG. 4B) were lowered by both doses of tolfenamic acid as shown in FIGS. 4A and 4B. Tau phosphorylated at Ser 235 was lowered by about 15% as indicated by one-way ANOVA (F(2,11)=6.105, p=0.0165), Tukey-Kramer post-test p<0.05 for the C vs the 5 mg/kg group and for the C vs the 50 mg/kg group. P-tau at Thr 181 was lowered by about 30%, one-way ANOVA F(2,10)=7.272, p=0.0112, Tukey-Kramer post-test p<0.05 for the C vs the 5 mg/kg group and for the C vs the 50 mg/kg group.

### EXAMPLE 4

### Tolfenamic Acid Improves Learning and Memory in APP YAC AD Mouse Model

In order to examine the ability of tolfenamic acid to improve the learning and memory deficits in AD transgenic mice model, male and female homozygous APP YAC transgenic, line R1.40, and wild type C57BL/6 mice aging between 20-24 months were used. Four groups of similar age and gender variation were administered vehicle or tolfenamic acid daily via oral gavages for 34 days; one transgenic group was administered 5 mg/kg/day tolfenamic acid in corn oil (n=7); the second transgenic group was administered 50 mg/kg/day tolfenamic acid in corn oil (n=7), the third transgenic group was administered corn oil, the vehicle, and the last group which consisted of wild type C57BL/6 mice was administered vehicle (n=8).

After 14 days of daily dosing, mice were tested in Morris water maze. In this task, the mice had to locate the hidden platform by learning multiple spatial relationships between the platform and the distal extra-maze cues. On the first day, mice received a habituation trial in which they were allowed to swim freely for 60 seconds. On the following day and for a total of 8 days, mice received training sessions of 3 trials daily. The starting position for each trial was randomly assigned between the four possible positions while the platform position was fixed in each trial. Each animal was allowed to swim until they find the immersed hidden platform or for a maximum of 60 seconds.

Upon conducting the 8 acquisition sessions, probe trials on Day 1 and Day 11 following the last day of training were performed to assess the long-term memory retention by studying the preference of the mice to the correct quadrant that contained the hidden platform. On day 33, mice were tested for spontaneous alternation in Y-maze, defined as the percentage of the number of arm entries different from the previous two entries divided by the total arm entries minus two. Finally, on Day 35, mice were euthanized and brain tissue was removed and stored at −80° C.

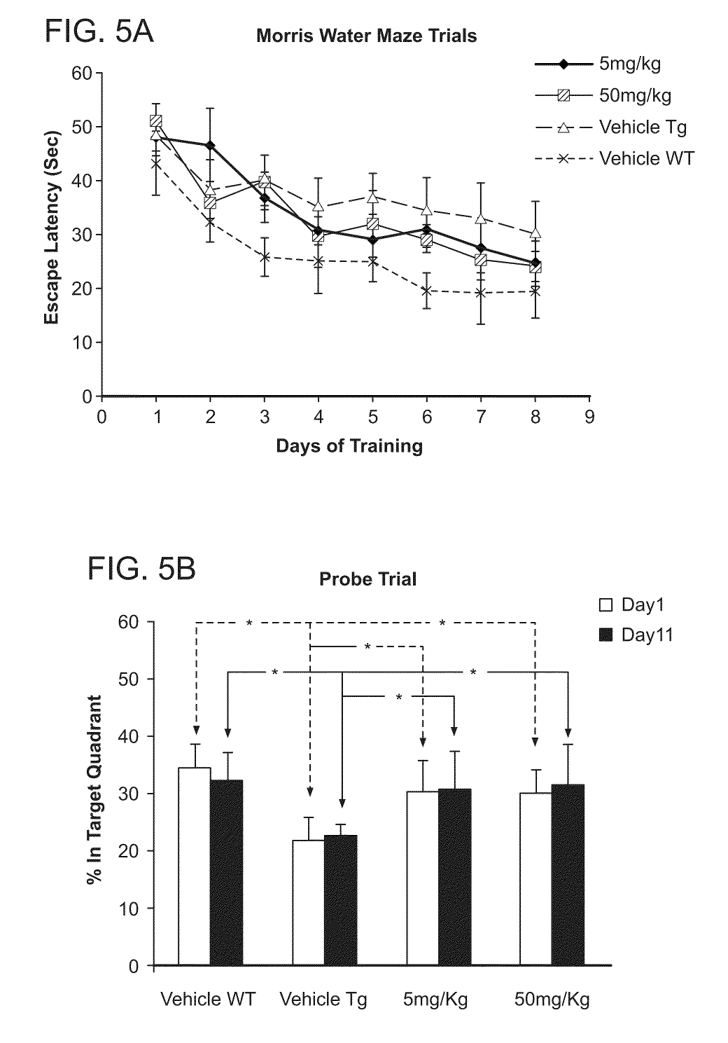

Analysis of the results indicated a decline in spatial learning of the transgenic group when compared to the wild type C57BL/6 group and that profile was improved following administration of both doses of tolfenamic acid (as shown in FIG. 5A). In addition, the probe trials on Day 1 and Day 11 following the 8 daily training sessions showed significant impaired long-term memory abilities in the transgenic group that improved significantly upon administration of 5 and 50 mg/kg/day tolfenamic acid where mice showed more preference for the correct quadrant (as shown in FIG. 5B).

In FIG. 5A, the daily trials showed a different acquisition pattern when the transgenic and the wild type C57BL/6 group were compared. The transgenic group had a higher mean escape latency indicating a deficit in their learning ability to locate the hidden platform that was improved with tolfenamic acid administration. In FIG. 5B, the analysis of the long-term memory retention profile revealed memory impairments in the transgenic group and treatment with 5 and 50 mg/kg/day tolfenamic acid resulted in a significant increase in the % time spent in the target quadrant at Day 1 and 11 following the 8 daily training sessions indicating an improvement in their long-term memory retention abilities. Again, the symbol “*” indicates that values are significantly different from control, as determined by a Student's t-test (p<0.05).

### EXAMPLE 5

### Tolfenamic Acid Improves the Working Memory Function

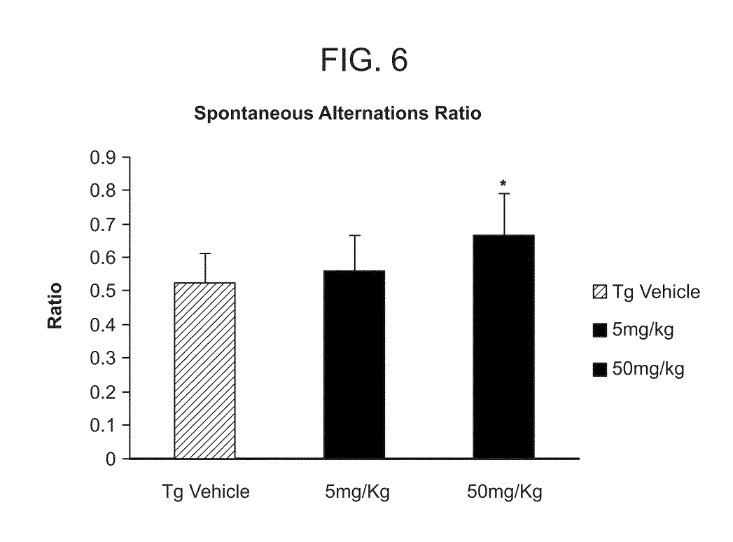

For the first time, it has been shown that tolfenamic acid treatment significantly increased the spontaneous alternation ratio in the Y-maze indicating an improvement in the working memory function of the treated groups (as shown for example, in FIG. 6). Mice treated with tolfenamic acid showed an increase in the spontaneous alternation ratio that was significant in the group treated with 50 mg/kg/day. Again, the symbol “*” indicates that values are different from control, as determined by a Student's t-test (p<0.05).

The improvement in the behavioral profile after administration of 5 mg/kg/day tolfenamic acid did not reach significance, and it is probable that an increase in the duration of tolfenamic acid treatment with such dose could improve the memory deficits identified by this test significantly. It has been found, therefore, that the treatment reduces tau gene expression in the brain by at least 45% and more preferably by at least 50% (46% and 48% have been shown), and that the treatment lowers phosphorylated tau protein in the brain by at least 15%-30%. The effective dose that was administered to the subject was 1 mg to 50 mg of per kg of weight of the patient.

Those skilled in the art will appreciate that numerous modifications and variations may be made to the above disclosed embodiments without departing from the spirit and scope of the present invention.

What is claimed is:

1. A method of reducing the level of tau protein in a subject's brain for the treatment of tau tangles, the method comprising administrating an effective dose of drug that reduced Sp1 levels to the subject to lower tau mRNA and protein levels, CDK5 mRNA and protein levels, as well phosphorylated tau in the brain.

2. The method of claim 1, wherein the drug is tolfenamic acid or analogs thereof.

3. The method of claim 1, wherein the treatment reduces tau gene expression in the brain by at least 45%.

4. The method of claim 1, wherein the treatment lower tau protein in the brain by about 46%.

5. The method of claim 1, wherein the treatment reduces tau gene expression in the brain by about 48%.

6. The method of claim 1, wherein the treatment reduces tau gene expression in the brain by at least 50%.

7. The method of claim 1, wherein the treatment lowers CDK5 mRNA and protein levels protein in the brain by at least 40-50%.

8. The method of claim 1, wherein the treatment lower phosphorylated tau protein in the brain by at least 15-30%.

9. The method of claim 1, wherein the effective dose is administered to the subject is 1 to 50 mg of per kg of weight of patient.

10. The method of claim 1, wherein the treatment improved learning and memory in the subject.

11. The method of claim 1, wherein the treatment improved working memory function in the subject.
